(19) United States
(12) Patent Application Publication
(10) Pub. No.: US 2023/0381456 A1
(43) Pub. Date: Nov. 30, 2023

(54) CATHETER EXCHANGE STRATEGY AND CATHETER CONFIGURED FOR SAME

(71) Applicant: INMed, Inc., Plainfield, IN (US)

(72) Inventors: Thomas P. LaRoche, Danville, IN (US); Jeffrey R. Ramkaransingh, Carmel, IN (US)

(73) Assignee: INMed, Inc., Plainfield, IN (US)

(21) Appl. No.: 18/198,912

(22) Filed: May 18, 2023

Related U.S. Application Data
(60) Provisional application No. 63/346,563, filed on May 27, 2022.

(51) Int. Cl. A61M 25/01; A61J 15/00; A61M 39/02

(52) U.S. Cl. CPC A61M 25/0113; A61J 15/0015; A61M 39/0247; A61J 15/0069

(57) ABSTRACT

Exchanging a catheter includes producing a first image on an electronic display establishing positioning of a first catheter at a treatment location within a patient where a distal tip of the first catheter remains below the pylorus in a patient's gastro-intestinal (GI) tract. An exchange wire is advanced through the first catheter to a predefined target insertion depth, and the first catheter is removed from the patient over the exchange wire. A replacement catheter is then advanced over the common exchange wire, and a second image produced on an electronic display confirming placement of the second catheter at the treatment location. Aspects of the methodology can be performed without assistance of radiography and in a traditional emergency room setting.

### TECHNICAL FIELD

The present disclosure relates generally to exchanging a catheter in a patient, and more particularly to performing portions of a catheter exchange procedure without the assistance of radiography or other imaging.

### BACKGROUND

Catheters are used for a multiplicity of different medical treatments and interventional procedures in modern medicine. A typical catheter includes an elongate flexible body having one or more passages or lumens extending from a proximal end of the catheter outside of the patient to one or more treatment locations within the patient's body. The lumens can be used to pass a variety of liquid agents for imaging, nutrition, hydration, or medicinal treatment of a patient at targeted locations, as well as various interventional tools for establishing or reestablishing access within a patient's vasculature or other structures within a patient's body.

Typical catheter placement includes advancing a wire guide into the patient's body and tracking a catheter over the wire guide to a desired location. Some techniques involve placement of the catheter only briefly until a specific procedure can be completed, followed by withdrawal. Other interventions require the catheter to remain within the patient for a more extended period of time. In one example, a catheter known generally in the art as a gastro jejunal or “GJ” feeding tube is advanced into a patient's gastro-intestinal or “GI” tract with a first feeding port in the catheter placed in a patient's stomach and a second feeding port placed more distally, below the pylorus and in the patient's small intestine. Different nutrition and hydration substances can be delivered into the stomach versus the small intestine, at different times, or in different ways. Over the course of time it is common for so-called GJ tubes to become clogged, with material at least partially obstructing the feeding port(s). While efforts can be made to clear the obstruction, it is commonly desirable to simply swap out the GJ tube for a replacement. Proper placement of a GJ tube can be challenging, however, and in some instances the GJ tube, whether clogged or not, can become displaced from a desired treatment location.

Catheter exchange techniques are known where one catheter is swapped out for another catheter using a common wire guide advanced through the first catheter and then maintained in place while the first catheter is removed and a replacement catheter advanced over the wire guide. While such techniques can be successful, it is conventional to perform such procedures with the assistance of live radiography. Suites for live radiography in a hospital setting often need to be reserved in advance, and thus scheduling a catheter exchange or other procedures can be administratively burdensome. One known GJ tube apparatus is set forth in co-pending and commonly owned U.S. patent application Ser. No. 17/767,747, filed Apr. 8, 2022, and a National Stage Application of PCT/US22/13325.

### SUMMARY

In one aspect, a method of exchanging a catheter includes producing a first image on an electronic display establishing positioning of a first catheter at a treatment location within a patient, and advancing an exchange wire through the first catheter to a predefined target insertion depth. The method further includes removing the first catheter from the patient, and advancing a replacement catheter over the exchange wire. The method still further includes producing a second image on an electronic display confirming placement of the second catheter at the treatment location within the patient.

In another aspect, a method of swapping out a gastro-jejunal (GJ) feeding tube includes confirming, via fixed radiography, a jejunal feeding tip of a first GJ tube within a patient remains below the pylorus in a patient's gastro-intestinal (GI) tract. The method further includes swapping the first GJ tube for a replacement GJ tube over a common exchange wire without radiography. The method still further includes confirming, via fixed radiography, a jejunal feeding tip of the replacement GJ tube within the patient is below the pylorus in the patient's GI tract.

In still another aspect, a catheter includes an elongate tubular body defining a longitudinal axis and including a proximal end, a distal tip having a distal feed opening formed therein, and a medial feed opening formed axially between the proximal end and the distal tip. The elongate tubular body further includes a first feed lumen extending to the distal feed opening, a second feed lumen extending to the medial feed opening, and a normally closed retrieval lumen originating at an origination location axially between the medial feed opening and the distal tip and terminating at a termination location distal to the origination location. The catheter further includes a transducer within the distal tip.

### BRIEF DESCRIPTION OF THE DRAWINGS

FIG. 1 is a diagrammatic view of a catheter package, including a detailed enlargement, according to one embodiment;

FIG. 2 is a sectioned side diagrammatic view of a portion of a catheter, according to one embodiment;

FIG. 3 is a sectioned side diagrammatic view of a distal tip portion of a catheter and an imaging system, according to one embodiment;

FIG. 4 is a sectioned side diagrammatic view of a portion of a catheter at one stage of an exchange procedure, according to one embodiment;

FIG. 5 is a diagrammatic view, including a detailed enlargement, of a catheter and exchange wire at another stage of an exchange procedure, according to one embodiment;

FIG. 6 is a diagrammatic view of a catheter at one stage of an exchange procedure, according to one embodiment; and

FIG. 7 is a diagrammatic view of a catheter at another stage of an exchange procedure, according to one embodiment.

### DETAILED DESCRIPTION

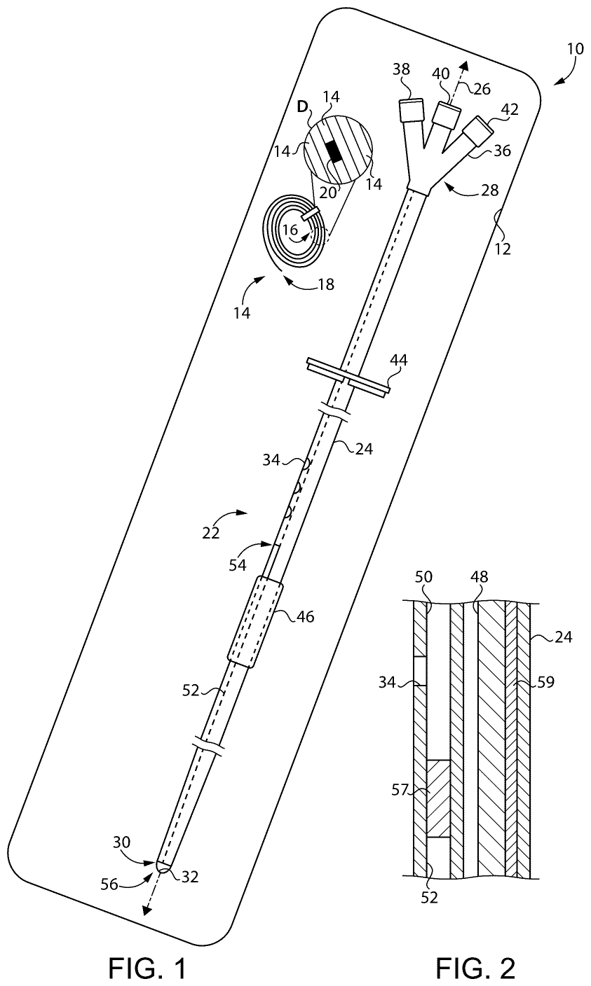

Referring to FIG. 1, there is shown a catheter package 10, according to the present disclosure and including a sterile envelope 12, and an exchange wire 14 and a catheter 22 sealed within sterile envelope 12. Exchange wire 14 is an elongate wire guide formed of stainless steel, nitinol, or various other metallic alloys, and having a proximal end 16 and a distal end 18 and is stored in a coiled-up configuration within sterile envelope 12. Catheters might be packaged separately from wire guides in various embodiments. Exchange wire 14 also includes an indelible indicator 20 thereon. Indelible indicator 20 may be closer to proximal end 16 than to distal end 18 in some embodiments, and a functionality further discussed herein for establishing advancement through a catheter for catheter exchange purposes to a predefined target insertion depth. As shown in the detailed enlargement D, indelible indicator 20 may include a marking such as a color marking, a shade marking, a wrapped piece of colored tape, a surface texture, a coating, an insert of a material different than a catheter body material, or any other configuration that can be used by a clinician to visually monitor an insertion depth of exchange wire 14 in catheter 22 as further discussed herein.

Catheter 22 includes an elongate tubular body 24 defining a longitudinal axis 26 and including a proximal end 28, a distal tip 30 having a distal feed opening 32 formed therein, and a medial feed opening 34 formed axially between proximal end 28 and distal tip 30. Referring also now to FIG. 2, elongate tubular body 24 further includes a first feed lumen 48 extending to distal feed opening 32, a second feed lumen 50 extending to medial feed opening 34, and a normally closed retrieval lumen 52. Catheter 22 also includes a hub 36 having a plurality of feed ports formed therein including a first feed port 38 that may fluidly connect to first feed lumen 48, a second feed port 40 that may fluidly connect to second feed lumen 50, and a third port 42. Third port 42 may include a balloon inflation port fluidly connecting to a balloon attached to or formed integrally with elongate tubular body 24. A plurality of feed lumens and a balloon port may or may not be used in some implementations, and catheter 22, namely elongate tubular body 24, will typically include at least one lumen extending entirely therethrough. Catheter 22 may be a so-called gastro-jejunal (GJ) feed tube or catheter structured for providing hydration and nutrition, and potentially also imaging dye, medicaments, etc. to a patient's stomach and small intestine above and below, respectively, the pylorus or pyloric valve in a patient's gastro-intestinal (GI) tract as further discussed herein.

Retrieval lumen 52 originates at an origination location 54 axially between medial feed opening 34 and distal tip 30 and terminates at a termination location distal to origination location 54. Termination location 56 may be in distal tip 30 in some embodiments, although the present disclosure is not thereby limited. Elongate tubular body 24 also includes therein a plug 57 separating second feed lumen 50 from normally closed retrieval lumen 52 in the illustrated embodiments. This configuration allows plug 57 to be installed in a single lumen thereby dividing the lumen into two parts. In other embodiments, separate, parallel lumens might be used. Elongate tubular body 24 may also include therein an anti-cut structure 59. Anti-cut structure 59 can include an embedded metallic piece or sleeve or wire that limits a cut depth of a cutting tool such as a surgical scalpel through elongate tubular body 24 in a radial direction. Rather than a metallic material, anti-cut structure 59 could be formed of a relatively harder or tougher polymeric material such as a polyimide material. Elongate tubular body 24 may otherwise be formed predominantly of nylon in some embodiments. Catheter 24 may also include a disk 44 or other attached structure along elongate tubular body 24 that is to be positioned outside a patient and resists insertion of catheter 22 further into the patient's body than is desired. In addition, an anti-slip sleeve 46 is attached to elongate tubular body 24 and can, typically in cooperation with a securing clamp, disk, or other attached device, resist catheter 22 slipping into the patient's body when elongate tubular body 24 is cut for purposes further discussed herein. “Sleeve” 46 may or may not have a conventional sleeve-like structure and could be formed of a variety of materials or have a variety of shapes, including having a multiple parts. In some instances, a clamp releasably connectable to catheter 22 could perform the function of a sleeve. In an implementation, catheter 22 may have a structure substantially identical but for some differences disclosed herein to the catheter/GJ tube structure disclosed in commonly owned co-pending U.S. application Ser. No. 17/767,747, a National Stage application of PCT/US22/13325, filed Apr. 8, 2022, and hereby incorporated by reference.

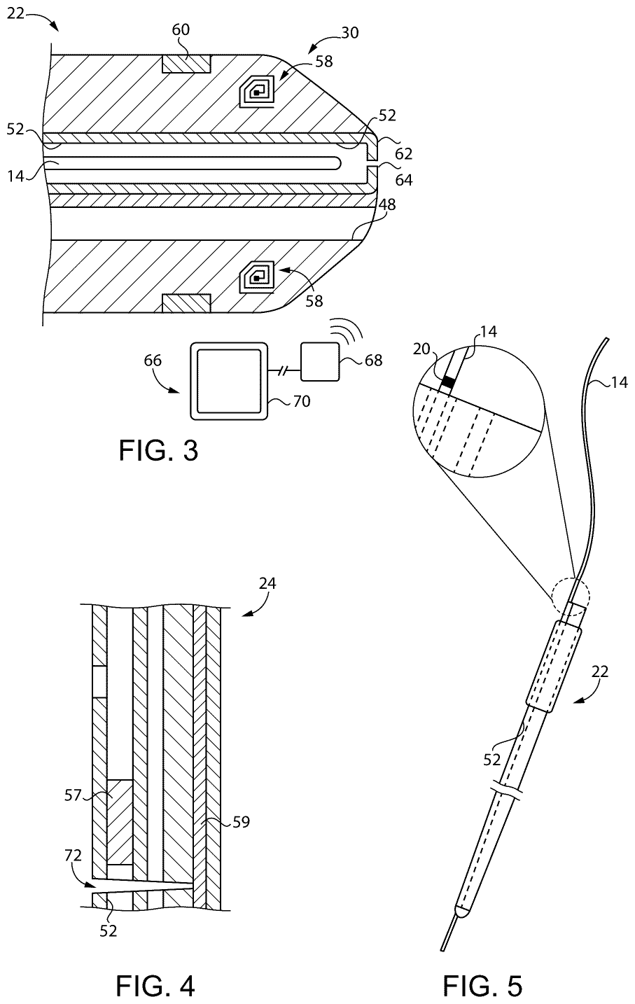

Referring also now to FIG. 3, there are shown additional features of catheter 22 in relation to distal tip 30. Distal tip 30 may include a distal terminal tip 62 with normally closed retrieval lumen 52 extending to a valve 64 located at distal terminal tip 62. Exchange wire 14 is shown within normally closed retrieval lumen 52 in FIG. 3 and can be advanced through valve 64 to open the same. One or more radiopaque embedded elements 60 may be within distal tip to assist with locating distal tip 30 during radiography where used. Catheter 22 may also include one or more transducers 58 within distal tip 30. Transducers 58 may include passive radiofrequency (RF) transducers or RF tags that produce or output RF signals when stimulated by way of an energizing RF signal source. In some embodiments transducers 58 can produce signals that can be used by way of a sensing array or sensor to produce, upon an electronic display screen, a visual indication of a location of distal tip 30 within a patient's body as further discussed herein. To this end, FIG. 3 also illustrates an imaging system 66 having an energizing signal source 68 structured to output an energizing signal that stimulates transducers 58. In other embodiments transducers 58 may output signals that are produced in response to electrically energizing the one or more transducers 58 by way of a wire or the like extending through elongate tubular body 24 and energized via an external electrical energy source. Embodiments may also include one or more transducers that are energized to generate a magnetic field(s) that is sensed to image or otherwise approximate catheter relative location in a patient's anatomy. Still other embodiments may include a signal producing transducer that is attached to a powered stylet advanced through a catheter. In such an embodiment a stylet in the form of a conductive wire having a transducer attached to the tip is positioned through a catheter and energized to generate an electromagnetic signal monitored by an external signal monitoring apparatus configured to produce a visually perceptible image that a clinician can use to monitor catheter placement within the patient's anatomy.

It should be appreciated the present disclosure is applicable without regard to the particular manner, frequency, or mechanism used to produce a signal from transducer(s) 58 that can be used to generate upon a display 70 a visual indication of a location or approximate location of distal tip 30 within a patient's anatomy. An image on an electronic display as contemplated herein includes a true image of a catheter or parts thereof within a patient's anatomy as well as various constructed images that are representative of catheter positioning. For instance, a constructed image of this nature could display a graph, a chart, an X-Y coordinate system, or an X-Y-Z coordinate system, and indicia or data reflecting relative catheter placement, to name a few examples. It is also contemplated that production of an image enabling visual monitoring of catheter location can occur via radiography. In a further implementation fixed radiography can be used for certain steps in a catheter exchange procedure and not used in other steps in a catheter exchange procedure.

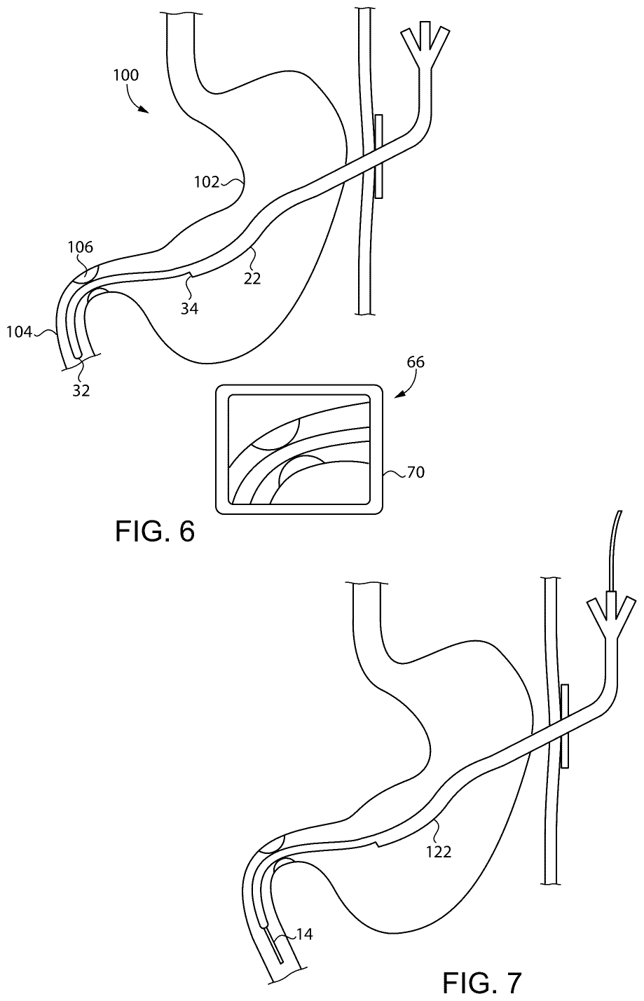

Referring also now to FIG. 4, there is shown elongate tubular body 24 as it might appear where having been cut partially through to establish access to normally closed retrieval lumen 52. A clinician has used a cutting tool and cut through catheter 24 up to anti-cut structure 59. From this stage catheter 22 can be manipulated to open access to normally closed retrieval lumen 52 to a wire guide, such as exchange wire 14 discussed above. FIG. 5 illustrates exchange wire 14 having been advanced through catheter 22 to a predefined target insertion depth, showing in a detailed enlargement that exchange wire 14 is inserted just to a point at which indelible marking 20 remains visible, the significance of which will be further apparent from the following description.

As explained above, it can be desirable to exchange or swap out one catheter for another, including the exchange of GJ tubes. Referring to FIG. 6, there is shown catheter 22 as it might appear in a patient's GI tract 100 where one or more medial feed openings 34 are within the patient's stomach 102 and distal feed opening 32 is within the patient's small intestine 104. Display 70 is also shown in FIG. 6 and produces a first image establishing positioning of a first catheter, catheter 22, at a treatment location within a patient. Producing the image as shown on electronic display 70 establishes the jejunal feeding tip (distal tip 30) of catheter 22 has not slipped back above the pylorus 106 in the patient's GI tract. Thus, catheter 22 might be a catheter that has been in service in a patient for some time and needs to be exchanged for a variety of reasons, but commonly exchanged for another catheter that is interchangeable for service with catheter 22 and which may be identical in many instances.

From the state depicted in FIG. 6, with confirmation that catheter 22 has not slipped back past (below) the pylorus, a clinician can assume that catheter 22 is properly placed to serve as a basis for placement of an exchange wire to be used for placement of a replacement catheter. Referring back to FIGS. 4 and 5, it will be recalled that elongate tubular body 24 can be cut partially or entirely to establish access to normally closed retrieval lumen 52. It can thus be readily visualized that from the state depicted in FIG. 6 exchange wire 14 is advanced through catheter 22, and then catheter 22 removed from the patient. As depicted in FIG. 7 a replacement catheter 122 is advanced over exchange wire 14. Prior to or after thenceforth removing exchange wire 14, a second image can be produced on electronic display 70 to confirm placement of second catheter 122 at the treatment location within the patient.

### INDUSTRIAL APPLICABILITY

As noted above, a variety of technologies are contemplated for producing images on electronic display 70, or any suitable electronic display, including radiography. In the clinical environment it can be inconvenient and time consuming to obtain scheduling and access to a live radiography suite. Access to a fixed X-ray machine can be, however, more readily and rapidly obtainable. The present disclosure contemplates exchanging catheters without the need for live radiography, and including producing the first image via fixed radiography that establishes the first catheter is at a treatment location within the patient, and then producing the second image confirming placement of the second catheter at the treatment location with fixed radiography. Intervening aspects of the disclosed methodology, including swapping a first catheter such as a GJ feed tube for a replacement GJ feed tube, can occur without radiography altogether. Accordingly, swapping out a catheter according to the present disclosure can be achieved by way of equipment and procedures available in a traditional emergency room environment. By providing an indelible indicator on exchange wire 14 a clinician can perform the entire procedure by first obtaining a fixed radiograph via a conventional X-ray machine, swapping the catheters without any imaging whatsoever, and then confirming correct placement again utilizing the traditional X-ray equipment. This strategy is contemplated to provide significant advantages respecting availability or lack of availability, and/or cost, of live radiography equipment as well as related administratively burdensome healthcare procedures.

The present description is for illustrative purposes only, and should not be construed to narrow the breadth of the present disclosure in any way. Thus, those skilled in the art will appreciate that various modifications might be made to the presently disclosed embodiments without departing from the full and fair scope and spirit of the present disclosure. Other aspects, features and advantages will be apparent upon an examination of the attached drawings and appended claims. As used herein, the articles “a” and “an” are intended to include one or more items, and may be used interchangeably with “one or more.” Where only one item is intended, the term “one” or similar language is used. Also, as used herein, the terms “has,” “have,” “having,” or the like are intended to be open-ended terms. Further, the phrase “based on” is intended to mean “based, at least in part, on” unless explicitly stated otherwise.

What is claimed is:

1. A method of exchanging a catheter comprising:
producing a first image on an electronic display establishing positioning of a first catheter at a treatment location within a patient;
advancing an exchange wire through the first catheter to a predefined target insertion depth;
removing the first catheter from the patient;
advancing a replacement catheter over the exchange wire; and
producing a second image on an electronic display confirming placement of the second catheter at the treatment location within the patient.

2. The method of claim 1 wherein the producing a first image includes producing a first fixed radiographic image, and the producing a second image includes producing a second fixed radiographic image.

3. The method of claim 1 further comprising confirming advancement of the exchange wire to the predefined target insertion depth via an indelible indicator upon at least one of the exchange wire or the first catheter.

4. The method of claim 3 wherein the indelible indicator includes a marking upon the exchange wire.

5. The method of claim 3 wherein the first catheter and the second catheter are interchangeable for service at the treatment location within the patient.

6. The method of claim 5 wherein the treatment location is in the patient's gastro-intestinal (GI) tract.

7. The method of claim 6 wherein each of the first catheter and the second catheter includes a gastro jejunal (GJ) feeding tube.

8. The method of claim 7 wherein the producing the first image includes producing the first image establishing a jejunal feeding tip of the first catheter has not slipped back above the pylorus in the patient's GI tract.

9. The method of claim 1 further comprising establishing access for the exchange wire through the first catheter at a location between a proximal end and a distal end of the first catheter.

10. The method of claim 9 wherein the establishing access includes forming an opening to a normally closed retrieval lumen of the catheter.

11. The method of claim 10 wherein the forming an opening to the normally closed retrieval lumen includes cutting at least partially through the first catheter.

12. The method of claim 11 further comprising limiting a cut depth radially through the first catheter via an embedded anti-cut structure of the first catheter.

13. The method of claim 1 further comprising activating a transducer embedded in a tip of the first catheter to produce a signal, and the producing a first image includes producing the first image based on the signal.

14. A method of swapping out a gastro jejunal (GJ) feeding tube comprising:
confirming, via fixed radiography, a jejunal feeding tip of a first GJ tube within a patient remains below the pylorus in a patient's gastro-intestinal (GI) tract;
swapping the first GJ tube for a replacement GJ tube over a common exchange wire without radiography; and
confirming, via fixed radiography, a jejunal feeding tip of the replacement GJ tube within the patient is below the pylorus in the patient's GI tract.

15. The method of claim 14 wherein the swapping the first GJ tube for the replacement GJ tube includes advancing the common exchange wire through the first GJ tube to a predefined target insertion depth, and further comprising confirming the advancement of the common exchange wire to the predefined target insertion depth via an indicator upon the common exchange wire.

16. The method of claim 15 wherein the indicator includes an indelible indicator.

17. A catheter comprising:
an elongate tubular body defining a longitudinal axis and including a proximal end, a distal tip having a distal feed opening formed therein, and a medial feed opening formed axially between the proximal end and the distal tip;
the elongate tubular body further including a first feed lumen extending to the distal feed opening, a second feed lumen extending to the medial feed opening, and a normally closed retrieval lumen originating at an origination location axially between the medial feed opening and the distal tip and terminating at a termination location distal to the origination location; and
a transducer within the distal tip.

18. The catheter of claim 17 wherein the transducer includes a wireless transducer.

19. A catheter assembly including the catheter of claim 17 and an exchange wire having an indelible indicator positioned to establish a target insertion depth of the exchange wire through the catheter when the exchange wire is advanced through the catheter.
